(19) United States
(12) Patent Application Publication
(10) Pub. No.: US 2007/0196398 A1
(43) Pub. Date: Aug. 23, 2007

(54) Fluoroquinolone fatty acid salt compositions

(76) Inventor: Yerramilli V. S. N. Murthy, Apex, NC (US)

Correspondence Address: KENYON & KENYON LLP, 1500 K STREET N.W., SUITE 700, WASHINGTON, DC, 20005, US

(21) Appl. No.: 11/356,143

(22) Filed: Feb. 17, 2006

(51) Int. Cl. A61K 31/496; A61K 31/4709

(52) U.S. Cl. 424/400; 514/253.08; 514/312

(57) ABSTRACT

The invention relates to pharmaceutical compositions of a fatty acid salt of a fluoroquinolone and to methods of treating a condition in an animal by administering to an animal in need thereof the pharmaceutical composition of the invention.

### CROSS REFERENCE TO RELATED APPLICATIONS

Not Applicable.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### FIELD OF THE INVENTION

The invention relates to fluoroquinolone containing pharmaceutical compositions and to methods of treating a condition in an animal by administering to an animal in need thereof the pharmaceutical composition of the invention. The pharmaceutical compositions comprise a fatty acid salt of a fluoroquinolone and one or more pharmaceutically acceptable excipients. In one embodiment, the pharmaceutical compositions are a suspension of a fatty acid salt of a fluoroquinolone in an oil.

### BACKGROUND OF THE INVENTION

The following discussion of the background of the invention is merely provided to aid the reader in understanding the invention and is not admitted to describe or constitute prior art to the present invention.

Fluoroquinolones are an antibiotic used to treat infections caused by microorganisms. Fluoroquinolones have the basic structure shown below: 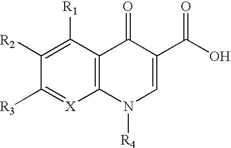
wherein R₁, R₂, R₃, and R₄ can be a variety of functional groups and X can be carbon or a variety of heteroatoms either of which may be substituted or unsubstituted.

Fluoroquinolones were first developed in the early 1960s. The first fluoroquinolone, nalidixic acid, was approved by the FDA in 1963 for the treatment of urinary tract infections. Nalidixic acid is rapidly absorbed after oral administration and is excreted into the urine in bactericidal concentrations. Nalidixic acid, however, has several limitations that prevents its use in other types of infections. Specifically, nalidixic acid has a narrow spectrum of activity and microorganisms easily developed resistance to the drug. The development of other fluoroquinolones by chemically altering the basic structure of nalidixic acid, however, has led to improved fluoroquinolone that are more effective against resistant bacteria and effective against a broader range of bacteria.

Representative fluoroquinolones include, but are not limited to, those described in BE 870,576, U.S. Pat. No. 4,448,962, DE 3,142,854, EP 047,005, EP 206,283, BE 887,574, EP 221,463, EP 140,116, EP 131,839, EP 154,780, EP 078,362, EP 310,849, EP 520,240, U.S. Pat. No. 4,499,091, U.S. Pat. No. 4,704,459, U.S. Pat. No. 4,795,751, U.S. Pat. No. 4,668,784, and U.S. Pat. No. 5,532,239.

The fluoroquinolone class of antibiotics are a powerful tool in combating bacterial infections. Fluoroquinolones have been used extensively to treat respiratory tract infections (including for example, bronchitis, pneumonia, tuberculosis), urinary tract infections, diarrhea, postoperative-wound infections, bone and joint infections, skin infections, inflammation of the prostate, ear infections, various sexually transmitted diseases, various infections that affect people with AIDS, and other conditions, in animals and humans. Fluoroquinolone are active against a wide spectrum of gram-positive and gram-negative bacteria. For example, various fluoroquinolones have been found to be effective against Staphylococcus aureus, Streptococcus pneumoniae, coagulese-negative staphylococci, Streptococcus pyogenes, Staphylococcus epidermis, Pseudomonas aeruginosa, Escherichia coli, Klebsiella pneumoniae, Enterobacter cloacae, Proteus mirabilis, Proteus vulgaris, Providencia stuartii, Morganella morganii, Citrobacter diversus, Citrobacter freundii, Haemophilus influenzae, and Neisseria gonorrhea, and other organisms. Indeed, the mounting resistance of Staphylococcus aureus to both penicillin and erythromycin has made the fluoroquinolone antibiotics a viable alternative for the treatment of skin diseases.

The mode of action of fluoroquinolones is to target DNA gyrase in the bacteria and interfere with bacterial replication.

Fluoroquinolones can be administered orally, topically, or parenterally.

U.S. Pat. No. 5,476,854 describes the oral, intravenous, and transdermal use of lomefloxacin to treat urinary tract infections, upper respiratory tract infections, sexually-transmitted infections, ophthalmologic infections, and intestinal infections.

U.S. Pat. No. 6,017,912 discloses a method of topically treating bacterial infections of the skin caused by susceptible organisms that comprises administering to an individual a composition of a fluoroquinolone antibiotic in a vehicle containing acetone and alcohol, applied directly to the affected areas of the human skin.

U.S. Pat. No. 5,912,255 discloses a pharmaceutical composition comprising a fluoroquinolone and benzoyl peroxide in a vehicle. The compositions are useful for the topical treatment of a variety of skin conditions.

U.S. Pat. No. 5,756,506 discloses a method of treating animals with fluoroquinolones by administering to the animal a single high dose of the fluoroquinolone to replace multiple lower doses.

U.S. Pat. No. 5,532,239 discloses treating nephrotic syndromes with fluoroquinolone derivatives.

U.S. Pat. No. 6,887,487 discloses compositions comprising a salt of a pharmacologically active compound and a lipophilic counterion and a pharmaceutically acceptable water soluble solvent combined together to form an injectable composition. The patent also discloses methods of treating a mammal with the disclosed compositions.

U.S. published application no. 2005/0049210 discloses composition for the administration of a pharmacologically active compound to a mammal comprising: a salt of the pharmacologically active compound with a lipophilic counterion; and a pharmaceutically acceptable, water immiscible solvent; combined together to form a composition that releases the active compound over time when administered to the mammal. The published application also discloses methods of treating a mammal with the disclosed compositions.

Solid oral pharmaceutical compositions, such as tablets and capsules, can be difficult for some individuals to swallow. For example, pediatric patients often find it difficult to swallow solid oral pharmaceutical compositions. Furthermore, solid oral pharmaceutical compositions can be difficult to administer to animals such as cats. Thus, in many instances liquid oral pharmaceutical compositions, such as solutions and suspensions, are desirable because they are easier to administer. Fluoroquinolones, however, are difficult to dissolve or suspend in liquids. Accordingly, there is a need in the art for new liquid fluoroquinolone compositions that can be more easily administered to animals.

Citation of any reference in this section of this application is not to be construed that such reference is prior art to the present application.

### SUMMARY OF THE INVENTION

The invention relates to a pharmaceutical composition comprising (i) a fatty acid salt of a fluoroquinolone and (ii) an excipient, wherein the pharmaceutical composition is adapted for oral administration.

In one embodiment, the pharmaceutical composition comprises a suspension of a fatty acid salt of a fluoroquinolone in an oil.

The invention further relates to a method of treating a condition in an animal comprising orally administering to the animal a pharmaceutical composition of the invention. The pharmaceutical compositions of the invention that are a suspension are in many instances easier to administer than solid oral dosage forms.

### BRIEF DESCRIPTION OF THE DRAWINGS

FIG. 1 is a graphical representation of the concentration of enrofloxacin in the serum in μg/mL as a function of time (hours) when enrofloxacin is administered to cats as a liquid pharmaceutical formulation of the invention (♦) and as a solid dosage form of commercially available enrofloxacin (i.e., Baytril®, commercially available from Bayer Health Care of Shawnee Mission, Kans.) (▪).

### DETAILED DESCRIPTION OF THE INVENTION

The invention relates to a pharmaceutical composition comprising (i) a fatty acid salt of a fluoroquinolone and (ii) an excipient, wherein the pharmaceutical composition is adapted for oral administration.

In one embodiment, the pharmaceutical composition comprises a suspension of a fatty acid salt of a fluoroquinolone in an oil.

The invention further relates to a method of treating a condition in an animal comprising orally administering to the animal a pharmaceutical composition of the invention.

### Definitions

The term “fatty acid salt of a fluoroquinolone,” as that term is used herein, means the salt formed between a fatty acid and a fluoroquinolone.

The term “fluoroquinolone,” as used herein, means any compound having the basic structure: 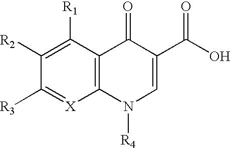
wherein R₁, R₂, R₃, and R₄ can be a variety of functional groups and X can be carbon, which may be substituted or unsubstituted, or nitrogen. One skilled in the art would readily recognize fluoroquinolones useful in the compositions and methods of the invention. Typically, the fluoroquinolones are useful as antibiotics but they may also be used to treat other conditions (for example, nephrotic syndromes).

The term “fatty acid,” as used herein, means a carboxylic acid of formula R—C(O)OH, wherein R is a C₆-C₂₂ hydrocarbon group. In one embodiment, R is a C₈-C₁₈ hydrocarbon group. In one embodiment, R is a C₁₀-C₁₈ hydrocarbon group.

The phrase a “C₆-C₂₂ hydrocarbon group,” as used herein means a straight or branched, saturated or unsaturated, cyclic or non-cyclic, aromatic or non-aromatic, carbocyclic or heterocyclic group having from 6 to 22 carbon atoms. Similarly, the phrases a “C₈-C₁₈ hydrocarbon group” and a “C₁₀-C₁₈ hydrocarbon group” means a straight or branched, saturated or unsaturated, cyclic or non-cyclic, aromatic or non-aromatic, carbocyclic or heterocyclic group having from 8 to 18 carbon atoms and from 10 to 18 carbon atoms, respectively. The hydrocarbon group can optionally be substituted with one or more groups selected from —OR₁; —COOR₁; —N(R₁)(R₁); —CON(R₁)(R₁); —SR₁; a C₅-C₆ carbocyclic or heterocyclic ring (wherein the heteroatom is selected from nitrogen, oxygen, and sulfur) optionally substituted with one or more groups selected from —OR₁; —COOR₁; —N(R₁)(R₁); —CON(R₁)(R₁); —SR₁, wherein R₁ is a C₁-C₄ straight chain or branched alkyl group.

The term “salt,” as used herein, means two compounds that are not covalently bound but are chemically bound by ionic interactions.

The term “oil,” as used herein, means a liquid ester of glycerol and three fatty acids, i.e., a triglyceride. The term “oil,” as used herein, also includes mineral oil. The term “mineral oil,” as used herein means an oil derived from a mineral source, such as petroleum, as opposed to oils derived from plants and animals. Representative sources of oil include, but are not limited to nuts, grains, seeds, fruits, vegetables, fish, and animals.

The term “pharmaceutically acceptable oil,” as used herein, means an oil that when administered to an animal does not have undue adverse effects such as excessive toxicity, irritation, or allergic response commensurate with a reasonable benefit/risk ratio.

The term “suspension,” as used herein, means solid particles that are evenly dispersed in a liquid and remain suspended with no visible signs of settling for at least about 2 minutes, preferably at least about 5 minutes, more preferably at least about 10 minutes, even more preferably at least about 15 minutes, and most preferably at least about 30 minutes. Accordingly, a suspension of a fatty acid salt of a fluoroquinolone in an oil is solid particles of the fatty acid salt of a fluoroquinolone that are evenly dispersed in a liquid and remain suspended with no visible signs of settling for at least about 2 minutes, preferably at least about 5 minutes, more preferably at least about 10 minutes, even more preferably at least about 15 minutes, and most preferably at least about 30 minutes.

The term “substantially free of,” as used herein, means less than about 2 percent by weight. For example, the phrase “a pharmaceutical composition substantially free of water” means that the amount of water in the pharmaceutical composition is less than about 2 percent by weight of the pharmaceutical composition,

The phrase “treating,” “treatment of,” and the like, as used herein, include the amelioration or cessation of a specified condition.

The phrase “preventing,” “prevention of,” and the like, as used herein, include the avoidance of the onset of a condition.

The term “condition,” as used herein means an interruption, cessation, or disorder of a bodily function, system, or organ.

The term “animal,” as used herein, includes, but is not limited to, humans, canines, felines, equines, bovines, ovines, porcines, amphibians, reptiles, and avians. Representative animals include, but are not limited to a cow, a horse, a sheep, a pig, an ungulate, a chimpanzee, a monkey, a baboon, a chicken, a turkey, a mouse, a rabbit, a rat, a guinea pig, a dog, a cat, and a human.

The term “effective amount,” as used herein, means an amount sufficient to treat or prevent a condition in an animal.

The term “about,” as used herein to describe a range of values, applies to both the upper limit and the lower limit of the range. For example, the phrase “ranges from about 0.9 to 2 equivalents of fatty acid” has the same meaning as “ranges from about 0.9 to about 2 equivalents of fatty acid.”

### The Pharmaceutical Compositions

The pharmaceutical composition comprise (i) a fatty acid salt of a fluoroquinolone and (ii) an excipient, wherein the pharmaceutical composition is adapted for oral administration.

In one embodiment, the pharmaceutical composition comprises (i) a fatty acid salt of a fluoroquinolone and (ii) an oil. The fatty acid salt of the fluoroquinolone is suspended in the oil, i.e., the pharmaceutical composition is a suspension of the fatty acid salt of a fluoroquinolone in the oil.

The fluoroquinolone can be any fluoroquinolone known to those skilled in the art. Representative fluoroquinolones useful in the compositions and methods of the invention include, but are not limited to, those described in BE 870,576, U.S. Pat. No. 4,448,962, DE 3,142,854, EP 047,005, EP 206,283, BE 887,574, EP 221,463, EP 140,116, EP 131,839, EP 154,780, EP 078,362, EP 310,849, EP 520,240, U.S. Pat. No. 4,499,091, U.S. Pat. No. 4,704,459, U.S. Pat. No. 4,795,751, U.S. Pat. No. 4,668,784, and U.S. Pat. No. 5,532,239, the contents of which are expressly incorporated herein by reference thereto.

Representative fluoroquinolones useful in the compositions and methods of the invention include, but are not limited to, ciprofloxacin (commercially available as Cipro®), enrofloxacin (commercially available as Baytril®), enoxacin (commercially available as Penetrex®), gatifloxacin (commercially available as Tequin®, gemifloxacin (commercially available as Factive®), levofloxacin (commercially available as Levaquin®), lomefloxacin (commercially available as Maxaquin®), moxifloxacin (commercially available as Avelox®), norfloxacin (commercially available as Noroxin®), ofloxacin (commercially available as Floxin®), sparfloxacin (commercially available as Zagam®), trovafloxacin (commercially available as Trovan®), difloxacin, cinofloxacin, pefloxacin, tosufloxacin, temafloxacin, fleroxacin, amifloxacin, binfloxacin, danofloxacin, marbofloxacin, ruflocaxin, and sarafloxacin.

In one embodiment, the fluoroquinolone is ciprofloxacin.

In one embodiment, the fluoroquinolone is enrofloxacin.

In one embodiment, the fluoroquinolone is gatifloxacin.

In one embodiment, the fluoroquinolone is gemifloxacin.

In one embodiment, the fluoroquinolone is levofloxacin.

In one embodiment, the fluoroquinolone is lomefloxacin.

In one embodiment, the fluoroquinolone is moxifloxacin.

In one embodiment, the fluoroquinolone is ofloxacin.

In one embodiment, the fluoroquinolone is sparfloxacin.

In one embodiment, the fluoroquinolone is trovafloxacin.

In one embodiment, the fluoroquinolone is difloxacin.

In one embodiment, the fluoroquinolone is cinofloxacin.

In one embodiment, the fluoroquinolone is pefloxacin.

In one embodiment, the fluoroquinolone is tosufloxacin.

In one embodiment, the fluoroquinolone is temafloxacin.

In one embodiment, the fluoroquinolone is fleroxacin.

In one embodiment, the fluoroquinolone is amifloxacin.

In one embodiment, the fluoroquinolone is binfloxacin.

In one embodiment, the fluoroquinolone is danofloxacin.

In one embodiment, the fluoroquinolone is marbofloxacin.

In one embodiment, the fluoroquinolone is ruflocaxin.

In one embodiment, the fluoroquinolone is sarafloxacin.

Any fatty acid known to those skilled in the art can be used in the compositions and methods of the invention.

In one embodiment, R in the fatty acid is a C₆-C₂₂ hydrocarbon group.

In one embodiment, R in the fatty acid is a C₈-C₁₈ hydrocarbon group. In one embodiment, R in the fatty acid is a C₁₀-C₁₈ hydrocarbon group.

In one embodiment, R in the fatty acid is a C₁₀-C₂₂ hydrocarbon group.

In one embodiment, R in the fatty acid is a C₆-C₂₂ hydrocarbon group that is straight or branched, saturated or unsaturated, and non-cyclic.

In one embodiment, R in the fatty acid is a C₈-C₁₈ hydrocarbon group that is straight or branched, saturated or unsaturated, and non-cyclic.

In one embodiment, R in the fatty acid is a C₁₀-C₁₈ hydrocarbon group that is straight or branched, saturated or unsaturated, and non-cyclic.

In one embodiment, R in the fatty acid is a C₁₀-C₂₂ hydrocarbon group that is straight or branched, saturated or unsaturated, and non-cyclic.

Representative fatty acids useful in the compositions and methods of the invention include, but are not limited to, caproic acid, lauric acid, myristic acid, palmitic acid, stearic acid, palmic acid, oleic acid, linoleic acid, and linolenic acid. In one embodiment, the fatty acid is lauric acid.

Without wishing to be bound by theory, it is believed that the fatty acid protonates an amino nitrogen atom of the fluoroquinolone to provide a protonated fluoroquinolone molecule and a fatty acid carboxylate anion. The protonated fluoroquinolone molecule and the fatty acid carboxylate anion interact ionically to form the fatty acid salt of a fluoroquinolone.

The pharmaceutical compositions of the invention are formulated in accordance with routine procedures as a composition adapted for oral administration to animals. Pharmaceutical compositions of the invention suitable for oral administration can be in the form of tablets, capsules, cachets, pills, lozenges, powders, granules, pastilles, as a solution or a suspension in an aqueous or non-aqueous liquid, as an oil-in-water or water-in-oil liquid emulsion, as an elixir or syrup, and the like, each containing a predetermined amount of the fatty acid salt of a fluoroquinolone as an active ingredient.

In solid dosage forms of the invention for oral administration (e.g., capsules, tablets, pills, dragees, powders, granules, and the like), the fatty acid salt of a fluoroquinolone is mixed with one or more pharmaceutically acceptable excipients, including, but not limited to: fillers or diluents, binders, disintegrants, and lubricants and, optionally, formed into the desired dosage form.

Representative fillers or diluents include, but are not limited to, lactose, sucrose, dextrose, glucose, sucrose, mannitol, propylene glycol, glycerin, mannitol, sorbitol, maltodextrin, and silicic acid.

Representative binders include, but are not limited to, cellulose and its derivatives (such as sodium carboxymethylcellulose, ethylcellulose, methylcellulose, microcrystalline cellulose, and hydroxypropylmethylcellulose), alginates, gelatin, polyvinyl pyrrolidone, magnesium aluminum silicate, starches (such as corn starch and potato starch), gelatin, tragacanth, carbomer, povidone, guar gum, xanthan gum, and acacia.

Representative disintegrating agents include, but are not limited to, agar-agar, calcium carbonate, potato or tapioca starch, alginic acid and the sodium salt thereof, certain silicates, effervescent mixtures, croscarmellose, crospovidone, sodium carboxymethyl starch, and sodium starch glycolate

Representative lubricants, include, but are not limited to, talc, calcium stearate, magnesium stearate, solid polyethylene glycols, and sodium lauryl sulfate.

Solid oral dosage forms can be prepared using any method known to those skilled in the art (See, for example, Remington's Pharmaceutical Sciences 1447-1676 (Alfonso R. Gennaro ed., 19th ed. 1995), incorporated herein by reference). For example, a tablet can be made by compression or molding. Typically, compressed tablets are prepared by blending a binder, lubricant, and inert diluent to form a free flowing powder and compressing the powder using a suitable machine. Molded tablets can be made by molding, in a suitable machine, a mixture of the powdered compound moistened with an inert liquid diluent. Tablets and pills can optionally be scored. Moreover, where in tablet or pill form, the compositions can be coated to delay disintegration and absorption in the gastrointestinal tract thereby providing a sustained action over an extended period of time

Solid compositions can also be employed as fillers in soft or hard-shelled gelatin capsules using, for example, excipients such as lactose or milk sugars, high molecular weight polyethylene glycols, and the like.

Solid dosage forms of the pharmaceutical compositions, such as tablets, dragees, capsules, pills and granules, can be prepared with coatings and shells, such as enteric coatings and other coatings well known in the pharmaceutical-formulating art. They can also be formulated to provide slow or controlled release of the fatty acid salt of the fluoroquinolone. For example, hydroxypropylmethyl cellulose in varying proportions can be used to provide a desired release profile. Other polymer matrices, liposomes and/or microspheres can also be used to provide a desired release profile. The solid oral dosage forms can also be formulated for rapid release, e.g., as a freeze-dried composition. The solid oral pharmaceutical compositions can also be formulated to release the fatty acid salt of the fluoroquinolone only, or preferentially, in a certain portion of the gastrointestinal tract, optionally, in a delayed manner. The fatty acid salt of the fluoroquinolone can also be in micro-encapsulated form, if appropriate, with one or more of the above-described excipients.

Liquid dosage forms for oral administration of the fatty acid salt of a fluoroquinolone include pharmaceutically acceptable emulsions, microemulsions, solutions, suspensions, syrups, and elixirs. In addition to the fatty acid salt of a fluoroquinolone, the liquid dosage forms can contain inert diluents commonly used in the art, including, but not limited to, water or other solvent(s) and solubilizing agents and emulsifiers, such as ethyl alcohol, isopropyl alcohol, ethyl carbonate, benzyl alcohol, benzyl benzoate, propylene glycol, 1,3-butylene glycol, oils, glycerol, tetrahydrofuryl alcohol, polyethylene glycols, fatty acid esters of sorbitan, and mixtures thereof.

Suspensions, in addition to the fatty acid salt of the fluoroquinolone, can contain suspending agents such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof. In one embodiment, the pharmaceutical composition is a suspension and is substantially free of suspending agents.

The orally administered compositions can also comprise a sweetening agent such as fructose, sucrose, xylitol, aspartame, saccharin, cyclamate and acsulfame.

The orally administered compositions can also comprise a flavoring agent such as peppermint, oil of wintergreen, bubble gum flavor, spearmint flavor, fruit flavors (such as cherry, grape, and orange), fish flavor such as tuna, and the like. A representative commercially available flavoring agent is Magnasweet (commercially available from Mafco of Camden N.J.).

The orally administered compositions can also comprise a coloring agent such as an FD&C dye.

The orally administered compositions can also comprise an antioxidant such as butylated hydroxyanisole, butylated hydroxytoluene, and vitamin E.

The orally administered compositions can also comprise a preserving agent such as phenolic compounds (such as phenol), alkyl esters of parahydroxybenzoic acid (such as methyl paraben, ethyl paraben, and propyl paraben), benzoic acid and the salts thereof, boric acid and the salts thereof, sorbic acid and salts thereof, chorbutanol, alcohols such as ethyl and benzyl alcohol, thimerosal, nitromersol, and quaternary ammonium compounds (such as benzalkonium chloride and cetylpyridinium chloride).

The oral pharmaceutical compositions can also further comprise humectants, such as glycerol; solution retarding agents, such as paraffin; absorption accelerators, such as quaternary ammonium compounds; wetting agents, such as, cetyl alcohol, glycerol monostearate, and non-ionic surfactants; absorbents, such as kaolin and bentonite clay; and buffering agents.

Examples of suitable pharmaceutical excipients are described in Remington's Pharmaceutical Sciences 1447-1676 (Alfonso R. Gennaro ed., 19th ed. 1995), incorporated herein by reference.

Typically, the fatty acid salt of fluoroquinolone is present in the pharmaceutical composition in an amount ranging from about 0.5 percent to 20 percent by weight of the pharmaceutical composition. In one embodiment, the fatty acid salt of fluoroquinolone is present in the pharmaceutical composition in an amount ranging from about 0.5 percent to 10 percent by weight of the pharmaceutical composition. In one embodiment, the fatty acid salt of fluoroquinolone is present in the pharmaceutical composition in an amount ranging from about 0.5 percent to 5 percent by weight of the pharmaceutical composition. In one embodiment, the fatty acid salt of fluoroquinolone is present in the pharmaceutical composition in an amount ranging from about 1 percent to 4 percent by weight of the pharmaceutical composition. In one embodiment, the fatty acid salt of fluoroquinolone is present in the pharmaceutical composition in an amount of about 2.3 percent by weight of the pharmaceutical composition. It is possible, however, to prepare pharmaceutical compositions wherein the fatty acid salt of fluoroquinolone is present in the pharmaceutical composition in an amount of up to about 30 percent by weight of the pharmaceutical composition and even higher. In one embodiment, the fatty acid salt of fluoroquinolone is present in the pharmaceutical composition in an amount ranging from about 20 percent to 30 percent by weight of the pharmaceutical composition.

The fatty acid salt of a fluoroquinolone can be prepared by contacting a fluoroquinolone and a fatty acid. Typically, the fluoroquinolone is contacted with the fatty acid by dissolving the fluoroquinolone in a suitable solvent to provide a solution and adding at least one equivalent of fatty acid per equivalent of fluoroquinolone to the resulting solution. Typically, the fatty acid is added to the solution with stirring. Typically, the solvent is at room temperature, however, the solvent can be heated to any temperature up to the boiling point of the solvent, provided that the elevated temperature does not cause decomposition of the fluoroquinolone or the fatty acid. Preferably, the solvent dissolves both the fluoroquinolone and the fatty acid. Generally, the solvent is an organic solvent. Preferably, the solvent is a non-aqueous solvent.

In one embodiment, the fatty acid salt of fluoroquinolone is prepared by dissolving a salt, other than a fatty acid salt (for example, a hydrochloride salt), of fluoroquinolone in a solvent to provide a solution; adding at least 2 equivalents of fatty acid to the solution; and then separating the resulting fatty acid salt of the fluoroquinolone from the solvent. Fluoroquinolones are typically commercially available as the hydrochloride salt or as the free base.

After the fatty acid is added to the solution, the resulting fatty acid salt of the fluoroquinolone is separated from the solution. In one embodiment, the fatty acid salt of the fluoroquinolone precipitates and is collected by filtration. In another embodiment, the solvent is removed by evaporation, typically under reduced pressure, to provide the fatty acid salt of a fluoroquinolone as a solid. Accordingly, it is preferable that the solvent is an organic solvent of low volatility so that it can be readily removed under reduced pressure. Suitable solvents useful for preparing the fatty acid salt of a fluoroquinolone include, but are not limited to, dichloromethane, methylene chloride, tetrahydrofuran, methanol, ethanol, acetone, ethyl acetate, and acetonitrile. In another embodiment, the fatty acid salt of the fluoroquinolone is prepared in a solvent that can dissolve the fatty acid salt of the fluoroquinolone (for example, dimethyl formamide or dimethyl sulfoxide) and, after the fatty acid salt of the fluoroquinolone is formed, water is added to the solvent to precipitate the fatty acid salt of the fluoroquinolone, which can then be collected by, for example, filtration.

The fatty acid salt of a fluoroquinolone can be purified using standard methods known to those skilled in the art including, but not limited to, recrystallization, extraction, and chromatography.

Typically, about 0.9 to 2, preferably about 0.95 to 1.5, more preferably about 1 to 1.3, and most preferably about 1 to 1.1 equivalents of fatty acid is used per equivalent of fluoroquinolone.

### Pharmaceutical Compositions Comprising a Fatty Acid Salt of a Fluoroquinolone and an Oil

In one embodiment, the pharmaceutical composition comprises (i) a fatty acid salt of a fluoroquinolone and (ii) an oil. The fatty acid salt of the fluoroquinolone is suspended in the oil, i.e., the pharmaceutical composition is a suspension of the fatty acid salt of a fluoroquinolone in the oil. The pharmaceutical compositions of the invention that are a suspension of the fatty acid salt of a fluoroquinolone in the oil are in many instances easier to administer than solid oral dosage forms.

Any oil known to one skilled in the art can be used in the compositions and methods of the invention. Preferably, the oil is a pharmaceutically acceptable oil.

Suitable oils useful in the compositions and methods of the invention include, but are not limited to, olive oil, avocado oil, cod liver oil, herring oil, salmon oil, sunflower oil, soybean oil, peanut oil, coconut oil, sesame oil, palm oil, corn oil, safflower oil, canola oil, grape seed oil, and mineral oil. Preferably, the oil is olive oil, safflower oil, soybean oil, or avocado oil.

Typically, the fatty acid salt of fluoroquinolone is present in the pharmaceutical composition in an amount ranging from about 0.5 percent to 20 percent by weight of the pharmaceutical composition. In one embodiment, the fatty acid salt of fluoroquinolone is present in the pharmaceutical composition in an amount ranging from about 0.5 percent to 10 percent by weight of the pharmaceutical composition. In one embodiment, the fatty acid salt of fluoroquinolone is present in the pharmaceutical composition in an amount ranging from about 0.5 percent to 5 percent by weight of the pharmaceutical composition. In one embodiment, the fatty acid salt of fluoroquinolone is present in the pharmaceutical composition in an amount ranging from about 1 percent to 4 percent by weight of the pharmaceutical composition. In one embodiment, the fatty acid salt of fluoroquinolone is present in the pharmaceutical composition in an amount of about 2.3 percent by weight of the pharmaceutical composition. It is possible, however, to prepare pharmaceutical compositions wherein the fatty acid salt of fluoroquinolone is present in the pharmaceutical composition in an amount of up to about 30 percent by weight of the pharmaceutical composition and even higher. In one embodiment, the fatty acid salt of fluoroquinolone is present in the pharmaceutical composition in an amount ranging from about 20 percent to 30 percent by weight of the pharmaceutical composition.

In one embodiment, the fatty acid salt of a fluoroquinolone is a salt formed from a fatty acid wherein R in the fatty acid is a C₁₀-C₁₈ hydrocarbon group and the fluoroquinolone is enrofloxacin, ciprofloxacin, marbofloxacin, or amifloxacin. In one embodiment, the fatty acid is lauric acid and the fluoroquinolone is enrofloxacin, ciprofloxacin, marbofloxacin, or amifloxacin.

In one embodiment, the fatty acid salt of a fluoroquinolone is a salt formed from a fatty acid wherein R in the fatty acid is a C₁₀-C₁₈ hydrocarbon group; the fluoroquinolone is enrofloxacin, ciprofloxacin, marbofloxacin, or amifloxacin, and the oil is olive oil, safflower oil, soybean oil, or avocado oil. In one embodiment, the fatty acid is lauric acid; the fluoroquinolone is enrofloxacin, ciprofloxacin, marbofloxacin, or amifloxacin; and the oil is olive oil, safflower oil, soybean oil, or avocado oil.

In one embodiment comprising an oil, the pharmaceutical composition of the invention is substantially free of water. In one embodiment, the pharmaceutical composition contains less than about 1 percent water by weight of the composition. In one embodiment, the pharmaceutical composition contains less than about 0.5 percent water by weight of the composition. In one embodiment, the pharmaceutical composition contains less than about 0.2 percent water by weight of the composition

The suspension of the fatty acid salt of a fluoroquinolone in the oil is prepared by adding the fatty acid salt of a fluoroquinolone to the oil and stirring or agitating the resulting mixture. For example, the mixture can be agitated using a vortex mixture or by sonication.

The fatty acid salt of a fluoroquinolone forms a suspension and the fatty acid salt of a fluoroquinolone remains suspended in the oil when the pharmaceutical composition is allowed to sit undisturbed. Typically, the fatty acid salt of fluoroquinolone remains suspended in the oil with no visible signs of settling for at least about 2 minutes. In one embodiment, the fatty acid salt of fluoroquinolone remains suspended in the oil with no visible signs of settling for at least about 3 minutes. In one embodiment, the fatty acid salt of fluoroquinolone remains suspended in the oil with no visible signs of settling for at least about 5 minutes. In one embodiment, the fatty acid salt of fluoroquinolone remains suspended in the oil with no visible signs of settling for at least about 10 minutes. In one embodiment, the fatty acid salt of fluoroquinolone remains suspended in the oil with no visible signs of settling for at least about 15 minutes. In one embodiment, the fatty acid salt of fluoroquinolone remains suspended in the oil with no visible signs of settling for at least about 30 minutes. In some cases the fatty acid salt of fluoroquinolone remains suspended in the oil with no visible signs of settling for at least about 1 day. In contrast, when a non-fatty acid salt of a fluoroquinolone, such as, for example, a hydrochloride salt, is added to an oil and agitated, the salt quickly settles to the bottom of the container rather than remaining suspended in the oil. In one embodiment, the fatty acid salt of a fluoroquinolone forms a suspension and the fatty acid salt of a fluoroquinolone remains suspended in the oil and shows no visible signs of settling when the pharmaceutical composition is allowed to sit undisturbed for a length of time that is longer than the length of time it takes for a similar pharmaceutical composition, except that the fatty acid salt of the fluoroquinolone is replaced with an equivalent amount of a hydrochloric acid salt of the fluoroquinolone, to show visible signs of settling.

The pharmaceutical compositions comprising a fatty acid salt of a fluoroquinolone and an oil can further comprise one or more pharmaceutically acceptable excipients. Suitable pharmaceutically acceptable excipients include, but are not limited to, sweeteners, flavoring agents, preservatives, and coloring agents, including but not limited to those described above. Such excipients are known in the art. Examples of suitable pharmaceutical excipients are described in Remington's Pharmaceutical Sciences 1447-1676 (Alfonso R. Gennaro ed., 19th ed. 1995), the contents of which are incorporated herein by reference.

### Methods of Treating or Preventing a Condition in an Animal

The invention further relates to methods of treating or preventing a condition in an animal comprising administering to an animal in need thereof an effective amount of a pharmaceutical composition of the invention.

In one embodiment, the invention relates to methods of treating a condition in an animal comprising administering to an animal in need thereof an effective amount of a pharmaceutical composition of the invention.

In one embodiment, the invention relates to methods of preventing a condition in an animal comprising administering to an animal in need thereof an effective amount of a pharmaceutical composition of the invention.

In one embodiment, the pharmaceutical composition is administered orally.

In one embodiment, the pharmaceutical composition is administered topically.

In one embodiment, the animal is a mammal.

In one embodiment, the animal is a canine, a feline, an equine, a bovine, an ovine, or a porcine.

In one embodiment the animal is a human.

In one embodiment, the animal is a non-human animal.

In one embodiment, the animal is a dog.

In one embodiment, the animal is a cat.

In one embodiment, the animal is a cow.

In one embodiment, the animal is a pig.

In one embodiment, the animal is a horse.

In one embodiment, the animal is a sheep.

In one embodiment, the animal is a monkey.

In one embodiment, the animal is a baboon.

In one embodiment, the animal is a rat.

In one embodiment, the animal is a mouse.

In one embodiment, the animal is a guinea pig.

Representative conditions that can be treated or prevented with the methods of the invention include, but are not limited to, bacterial infections and nephrotic syndromes (such as those disclosed in U.S. Pat. No. 5,532,239, the contents of which are expressly incorporated herein by reference thereto).

In one embodiment, the condition is a bacterial infection.

In one embodiment, the condition is a bacterial infection caused by Staphylococcus aureus, Streptococcus pneumoniae, coagulese-negative staphylococci, Streptococcus pyogenes, Staphylococcus epidermis, Pseudomonas aeruginosa, Escherichia coli, Klebsiella pneumoniae, Enterobacter cloacae, Proteus mirabilis, Proteus vulgaris, Providencia stuartii, Morganella morganii, Citrobacter diversus, Citrobacter freundii, Haemophilus influenzae, or Neisseria gonorrhea.

In one embodiment, the condition is a respiratory tract infection, a urinary tract infection, a postoperative-wound infection, a bone or joint infection, a skin infection, an ear infection, or a sexually transmitted disease.

In one embodiment, the condition is a nephrotic syndrome.

The effective amount administered to the animal depends on a variety of factors including, but not limited to the type of animal being treated, the condition being treated, the severity of the condition, and the specific fluoroquinolone being administered. One of ordinary skill in the art will readily know what is an effective amount of the pharmaceutical composition to treat a condition in an animal. For example when the fatty acid salt of a fluoroquinolone is a fatty acid salt of ciprofloxacin, the condition is a bacterial infection, and the animal is a dog, an effective amount is typically between about 2.5 and 10 mg of ciprofloxacin/lb (between about 5 and 30 mg of ciprofloxacin/kg) administered orally once a day or two times per day until 2-3 days after cessation of the condition. When the fatty acid salt of a fluoroquinolone is a fatty acid salt of ciprofloxacin, the condition is a bacterial infection, and the animal is a cat, an effective amount is typically not more than 5 mg of fluoroquinolone/kg administered orally once a day until 2-3 days after cessation of the condition.

In one embodiment, the effective amount of the pharmaceutical composition is administered orally once per day until 2-3 days after cessation of the condition.

In one embodiment, the effective amount of the pharmaceutical composition is administered orally as two doses per day until 2-3 days after cessation of the condition.

In one embodiment, effective amount of the pharmaceutical composition is administered orally once per day for 7 days.

In one embodiment, the effective amount of the pharmaceutical composition is administered orally as two doses per day for 7 days.

In one embodiment, the effective amount of the pharmaceutical composition is administered orally once per day for 14 days.

In one embodiment, the effective amount of the pharmaceutical composition is administered orally as two doses per day for 14 days.

In one embodiment, the effective amount of the pharmaceutical composition is administered orally once per day for 21 days.

In one embodiment, the effective amount of the pharmaceutical composition is administered orally as two doses per day for 21 days.

Administering a fluoroquinolone as a suspension of a fatty acid salt of the fluoroquinolone in an oil can be easier, especially when the animal is a non-human animal, than administering a solid oral dosage form such as a capsule or tablet. Similarly, administering a fluoroquinolone as a suspension of a fatty acid salt of the fluoroquinolone is easier than administering a fluoroquinolone parenterally.

The following examples are set forth to assist in understanding the invention and should not be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### EXAMPLES

### Example 1

### Pharmaceutical Composition of Enrofloxacin—Lauric Acid Salt in Avocado oil

3.59 gr of enrofloxacin and 4.2 gr of lauric acid were suspended in 100 ml of dichloromethane and stirred at room temperature to provide a clear solution. The solvent was removed under reduced pressure to provide a solid enrofloxacin-lauric acid salt. The resulting solid was then placed under vacuum created by a high vacuum pump to remove residual dichloromethane.

1.2473 gr of the above enrofloxacin-lauric acid salt was placed in a 25 ml volumetric flask and the flask filled to about 80% of its volume with avocado oil. The resulting mixture was sonicated to provide a uniform suspension. The volume of the flask was than made up to 25 ml with more avocado oil and mixed well to obtain the final pharmaceutical composition. The concentration of enrofloxacin in the resulting composition is about 23 mg/mL.

Similar compositions were made using olive oil and sunflower oil.

The above compositions can also be include a flavoring agent, such as mint flavor, to flavor the composition.

### Example 2

### Administration of Oral Enrofloxacin to Cats

1 mL of the pharmaceutical composition of Example 1 was administered to a cat. To each of another two cats was administered a single tablet (22.7 mg) of commercially available enrofloxacin (Baytril®, commercially available from Bayer Health Care of Shawnee Mission, Kansas). For each cat the concentration of enrofloxacin in the serum was determined as a function of time over a period of about 24 hours. Blood samples were obtained as a function of time and frozen.

For analysis, of the blood samples, the following procedure was followed:

1) Thaw sample completely and mix well;

2) Transfer 200 μl of the sample into a microfuge tube;

3) Add 400 μl of methanol and mix well;

4) Centrifuge at 13,000 rpm, −9° C. for 15 minutes;

5) Transfer the supernatant to a 20 mL scintillation vial and add 3,400 μl of mobile phase A (described below);

6) Mix well, filter using a Acrodisc 13 mm syringe filter with 0.2 μm membrane and analyze by HPLC using the following conditions:

Column: Waters X Bridge C-18 4.6 mm×50 mm column equipped with a
Gemini 4 mm×3 mm guard cartridge.

Injection Volume: 20 μL

Flow Rate: 2 mL/min., isochratic 85% mobile phase A
15% mobile phase B

Mobile Phase:
Composition:
   A: 100 mM phosphate buffer−pH 2.1
   B: Methanol

Acquisition Wavelength: 274 nm
Fluorescence: Excitation: 297 nm
   Emission: 440 nm
   Gain 1000
   Attenuation: 4

Run time: 10 min.

At the end of each analysis, the column is washed with 90 percent aqueous methanol.

Mobile phase A can be prepared by the following procedure:

1. Weigh 13.8 g of sodium phosphate monobasic monohydrate into a 1 liter beaker.

2. Add 500 mL of de-ionized water with stirring.

3. Adjust the pH to 2.11 with phosphoric acid.

4. Transfer the resulting solution into a 1 liter volumetric flask and fill with water to the mark and mix well.

The concentration of enrofloxacin was determined by comparing the enrofloxacin peak area obtained from HPLC analysis of the sample to a standard curve of peak area versus concentration of enrofloxacin obtained by HPLC analysis of several samples of known enrofloxacin concentration.

The standard curve was obtained by weighing 100 mg of commercially available enrofloxacin into a 100 mL volumetric flask and filling the flask to 100 mL with methanol to provide a 1 mg/mL enrofloxacin stock solution. The 1 mg/mL enrofloxacin stock solution was then diluted with methanol to provide various standards by adding 5 mL of the 1 mg/mL enrofloxacin stock solution to 500 mL of methanol to provide a 10 g/mL standard, adding 0.25 mL of the 1 mg/mL enrofloxacin stock solution to 100 mL of methanol to provide a 0.025 μg/mL standard, adding 0.5 mL of the 1 mg/mL enrofloxacin stock solution to 100 mL of methanol to provide a 0.05 μg/mL standard, adding 1 mL of the 1 mg/mL enrofloxacin stock solution to 100 mL of methanol to provide a 0.1 μg/mL standard, adding 3 mL of the 1 mg/mL enrofloxacin stock solution to 100 mL of methanol to provide a 0.3 μg/mL standard, adding 7 mL of the 1 mg/mL enrofloxacin stock solution to 100 mL of methanol to provide a 0.70 μg/mL standard, adding 15 mL of the 1 mg/mL enrofloxacin stock solution to 100 mL of methanol to provide a 1.5 μg/mL standard. 200 μL of blank serum was then placed in each of four microfuge tubes labeled standard for points 1, 2, 3, and 4. To the tubes labeled standard for points 1, 2, 3, and 4 was then added 200 μL of the 0.025 μg/mL standard, 0.05 μg/mL standard, 0.1 pg/mL standard, 0.3 μg/mL standard, respectively. 200 μL of methanol was then added to each of the tubes; the resulting mixtures mixed with a vortex mixture for about 20 seconds; and then centrifuged at 13,000 rpm at −9° C. for 15 minutes. The resulting supernatant of each sample was then transferred to individual 20 mL scintillation vial and 1400 mL of mobile phase A was added to each supernatant. Each resulting solution was then mixed well, filtered using a Acrodisc 13 mm syringe filter with 0.2 μm membrane and analyze by HPLC using the HPLC method described above to provide the standard curve.

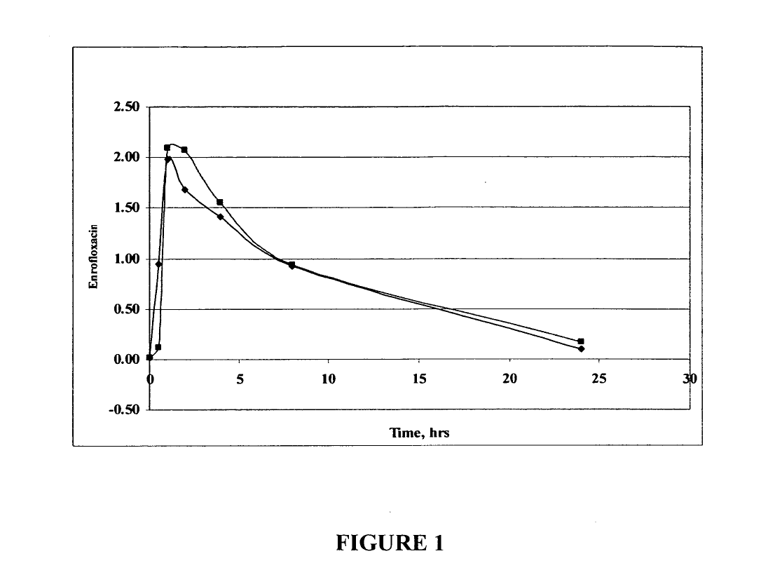

FIG. 1 is a graphical representation of the concentration of enrofloxacin in the serum as a function of time (hours). Each data point represents the serum concentration of enrofloxacin in μg/mL. The symbol (♦) represents data points for administration of enrofloxacin as the liquid pharmaceutical formulation of Example 1 in avocado oil. The symbol (▪) represents data points for administration of enrofloxacin as the solid dosage form of commercially available enrofloxacin (i.e., Baytril). Each data point ding to Baytril is the average serum concentration for the two cats. The data demonstrates that the oral liquid formulation is bioavailable.

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited, the entire disclosure of which are incorporated herein by reference.

What is claimed is:

1. A pharmaceutical composition comprising a suspension of a fatty acid salt of a fluoroquinolone in an oil.

2. The pharmaceutical composition of claim 1, wherein the fatty acid salt of a fluoroquinolone is a fatty acid salt formed between a fatty acid and a fluoroquinolone selected from the group consisting of ciprofloxacin, enrofloxacin, enoxacin, gatifloxacin, gemifloxacin, levofloxacin, lomefloxacin, moxifloxacin, norfloxacin, ofloxacin, sparfloxacin, trovafloxacin, difloxacin, cinofloxacin, pefloxacin, tosufloxacin, temafloxacin, fleroxacin, amifloxacin, binfloxacin, danofloxacin, marbofloxacin, ruflocaxin, and sarafloxacin.

3. The pharmaceutical composition of claim 1, wherein the fatty acid salt of a fluoroquinolone is a fatty acid salt formed between a fluoroquinolone and a fatty acid of formula RCOOH wherein R is a C₁₀-C₁₈ hydrocarbon group.

4. The pharmaceutical composition of claim 1, wherein the fatty acid salt of a fluoroquinolone is present in an amount ranging from about 0.5 percent to 20 percent by weight of the pharmaceutical composition.

5. The pharmaceutical composition of claim 4, wherein the fatty acid salt of a fluoroquinolone is present in an amount ranging from about 0.5 percent to 10 percent by weight of the pharmaceutical composition.

6. The pharmaceutical composition of claim 1, wherein the fatty acid salt of a fluoroquinolone is a fatty acid salt formed between a fluoroquinolone and a fatty acid selected from the group consisting of caproic acid, lauric acid, myristic acid, palmitic acid, stearic acid, palmic acid, oleic acid, linoleic acid, and linolenic acid.

7. The pharmaceutical composition of claim 6, wherein the fatty acid is lauric acid.

8. The pharmaceutical composition of claim 7, wherein the fluoroquinolone is selected from the group consisting of ciprofloxacin, enrofloxacin, amifloxacin, and marbofloxacin.

9. The pharmaceutical composition of claim 1, wherein the oil is selected from the group consisting of olive oil, avocado oil, cod liver oil, herring oil, salmon oil, sunflower oil, soybean oil, peanut oil, coconut oil, sesame oil, palm oil, corn oil, safflower oil, canola oil, grape seed oil, and mineral oil.

10. The pharmaceutical composition of claim 9, wherein the oil is selected from the group consisting of olive oil, safflower oil, soybean oil, and avocado oil.

11. The pharmaceutical composition of claim 9, wherein the fatty acid salt of a fluoroquinolone is a salt formed from a fatty acid of formula RCOOH, wherein R is a C₁₀-C₁₈ hydrocarbon group, and the fluoroquinolone is selected from the group consisting of enrofloxacin, ciprofloxacin, marbofloxacin, or amifloxacin.

12. The pharmaceutical composition of claim 11, wherein the fatty acid salt of a fluoroquinolone is present in an amount ranging from about 0.5 percent to 20 percent by weight of the pharmaceutical composition.

13. The pharmaceutical composition of claim 12, wherein the fatty acid salt of a fluoroquinolone is present in an amount ranging from about 0.5 percent to 10 percent by weight of the pharmaceutical composition.

14. The pharmaceutical composition of claim 11, wherein the fatty acid of formula RCOOH is lauric acid.

15. The pharmaceutical composition of claim 14, wherein the fatty acid salt of a fluoroquinolone is present in an amount ranging from about 0.5 percent to 20 percent by weight of the pharmaceutical composition.

16. The pharmaceutical composition of claim 15, wherein the fatty acid salt of a fluoroquinolone is present in an amount ranging from about 0.5 percent to 10 percent by weight of the pharmaceutical composition.

17. A method of treating or preventing a condition in an animal comprising orally administering to the animal an effective amount of the pharmaceutical composition of claim 1.

18. The method of claim 17, wherein the condition is a bacterial infection.

19. The method of claim 18, wherein the bacterial infection is caused by Staphylococcus aureus, Streptococcus pneumoniae, coagulese-negative staphylococci, Streptococcus pyogenes, Staphylococcus epidermis, Pseudomonas aeruginosa, Escherichia coli, Klebsiella pneumoniae, Enterobacter cloacae, Proteus mirabilis, Proteus vulgaris, Providencia stuartii, Morganella morganii, Citrobacter diversus, Citrobacter freundii, Haemophilus influenzae, and Neisseria gonorrhea.

20. The method of claim 17, wherein the condition is selected from the group consisting of a respiratory tract infection, a urinary tract infection, a postoperative-wound infection, a bone infection, a joint infection, a skin infection, an ear infection, and a sexually transmitted disease.

21. The method of claim 17, wherein the animal is selected from the group consisting of canines, felines, equines, bovines, ovines, porcines, amphibians, reptiles, and avians.

22. The method of claim 21, wherein the animal is selected from the group consisting of a cow, a horse, a sheep, a pig, an ungulate, a chimpanzee, a monkey, a baboon, a chicken, a turkey, a mouse, a rabbit, a rat, a guinea pig, a dog, a cat, and a human.

23. The method of claim 21, wherein the animal is a feline.

24. The method of claim 21, wherein the animal is a canine.

25. The method of claim 17, wherein the effective amount is administered once daily until 2-3 days after symptoms of the condition disappear.

26. A pharmaceutical composition comprising a suspension of a fatty acid salt of a fluoroquinolone, wherein said suspension takes longer to show visible signs of settling than a similar suspension of a hydrochloric acid salt of the fluoroquinolone.

27. The pharmaceutical composition of claim 26, wherein the fatty acid salt of a fluoroquinolone is a fatty acid salt formed between a fatty acid and a fluoroquinolone selected from the group consisting of ciprofloxacin, enrofloxacin, enoxacin, gatifloxacin, gemifloxacin, levofloxacin, lomefloxacin, moxifloxacin, norfloxacin, ofloxacin, sparfloxacin, trovafloxacin, difloxacin, cinofloxacin, pefloxacin, tosufloxacin, temafloxacin, fleroxacin, amifloxacin, binfloxacin, danofloxacin, marbofloxacin, ruflocaxin, and sarafloxacin.

28. The pharmaceutical composition of claim 26, wherein the fatty acid salt of a fluoroquinolone is a fatty acid salt formed between a fluoroquinolone and a fatty acid of formula RCOOH wherein R is a C₁₀-C₁₈ hydrocarbon group.

29. The pharmaceutical composition of claim 26, wherein the fatty acid salt of a fluoroquinolone is present in an amount ranging from about 0.5 percent to 20 percent by weight of the pharmaceutical composition.

30. The pharmaceutical composition of claim 26, wherein the fatty acid salt of a fluoroquinolone is a fatty acid salt formed between a fluoroquinolone and a fatty acid selected from the group consisting of caproic acid, lauric acid, myristic acid, palmitic acid, stearic acid, palmic acid, oleic acid, linoleic acid, and linolenic acid.

31. The pharmaceutical composition of claim 27, wherein the fluoroquinolone is selected from the group consisting of ciprofloxacin, enrofloxacin, amifloxacin, and marbofloxacin.

32. The pharmaceutical composition of claim 26, wherein the oil is selected from the group consisting of olive oil, avocado oil, cod liver oil, herring oil, salmon oil, sunflower oil, soybean oil, peanut oil, coconut oil, sesame oil, palm oil, corn oil, safflower oil, canola oil, grape seed oil, and mineral oil.

33. A method of treating or preventing a condition in an animal comprising orally administering to the animal an effective amount of the pharmaceutical composition of claim 26.

34. The method of claim 33, wherein the condition is a bacterial infection.

35. The method of claim 34, wherein the bacterial infection is caused by Staphylococcus aureus, Streptococcus pneumoniae, coagulese-negative staphylococci, Streptococcus pyogenes, Staphylococcus epidermis, Pseudomonas aeruginosa, Escherichia coli, Klebsiella pneumoniae, Enterobacter cloacae, Proteus mirabilis, Proteus vulgaris, Providencia stuartii, Morganella morganii, Citrobacter diversus, Citrobacter freundii, Haemophilus influenzae, and Neisseria gonorrhea.

36. The method of claim 33, wherein the condition is selected from the group consisting of a respiratory tract infection, a urinary tract infection, a postoperative-wound infection, a bone infection, a joint infection, a skin infection, an ear infection, and a sexually transmitted disease.

37. The method of claim 33, wherein the animal is selected from the group consisting of canines, felines, equines, bovines, ovines, porcines, amphibians, reptiles, and avians.

38. A pharmaceutical composition comprising (i) a fatty acid salt of a fluoroquinolone and (ii) a pharmaceutically acceptable excipient, wherein the pharmaceutical composition is adapted for oral administration.

39. The pharmaceutical composition of claim 38, wherein the pharmaceutical composition is in the form of a solid oral dosage form.

40. The pharmaceutical composition of claim 39, wherein the solid oral dosage form is selected from the group consisting of tablets, capsules, cachets, pills, lozenges, powders, granules, and pastilles.

41. The pharmaceutical composition of claim 38, wherein the pharmaceutical composition is in the form of a liquid oral dosage form.

42. The pharmaceutical composition of claim 41, wherein the liquid oral dosage form is selected from the group consisting of a solution, a suspension, an oil-in-water or water-in-oil liquid emulsion, an elixir, and a syrup.
